(19) United States
(12) Patent Application Publication
(10) Pub. No.: US 2012/0088668 A1
(43) Pub. Date: Apr. 12, 2012

(54) THINNING AGENT

(75) Inventors: Richard Williams, Colchester (GB); Tom Deckers, Bekkevoort (BE); Peter Roose, Ghent (BE); Johan Josef De Saegher, Ghent (BE)

(73) Assignee: TAMINCO, NAAMLOZE VENNOOTSCHAP, Ghent (BE)

(21) Appl. No.: 13/263,671

(22) PCT Filed: Apr. 7, 2010
(86) PCT No.: PCT/IB2010/000963

§ 371 Date: Dec. 27, 2011

(30) Foreign Application Priority Data
Apr. 7, 2009 (EP) 09447007.7

(51) Int. Cl. A01N 59/06; A01N 37/18; A01N 37/44; A01N 43/04; A01N 37/08; A01N 57/16; C07C 229/56; A01N 43/38

(52) U.S. Cl. 504/125; 562/458; 504/149; 504/339; 504/138; 504/140; 504/144; 504/127; 504/284; 504/291; 504/320; 504/196; 504/322

(57) ABSTRACT

A composition comprising a compound selected from an auxin, an auxin precursor, an auxin metabolite or a derivative of said auxin, auxin precursor or auxin metabolite or a mixture thereof and acetaminophen or a derivative thereof for use as a chemical thinning agent.

### FIELD OF THE INVENTION

This invention relates to a method and composition for reducing the number of fruits and/or flowers on plants by treating the plants with, particularly but not exclusively, anthranilic acid, optionally in combination with acetaminophen, or an auxin in combination with acetaminophen.

### BACKGROUND OF THE INVENTION

Many plants produce more fruits than is desirable for the production of high quality fruit. The thinning of fruits, or the blossoms that can lead to fruits, prevents this overproduction and can promote annual bearing. Thinning can also increase fruit size, improve fruit colour, and increase plant vigour.

Manual thinning is very expensive and labour intensive. Therefore there is a strong need for effective chemical thinners. Chemical thinners available to-date are sometimes not effective due to under thinning or over thinning and may be hazardous.

Naphthaleneacetic acid (NAA) is a commonly used fruit thinning agent. However, there is a risk of uncertainty in activity with this agent and it can result in foliar injury at high dose. Both NAA and another commercially available thinner, napthaleneacetimide (NAD), have been used in combination with the insecticide Carbaryl to increase their efficiency. However, Carbaryl, as an insecticide, is toxic to bees and to other beneficial insects.

Another commercial thinner is benzyladenine (BA). As with the use of NAA, pygmy fruits have been reported following its application with NAA or NAD.

Thus, the need remains for additional chemical thinning agents which may provide improved properties.

### SUMMARY OF THE INVENTION

The present invention relates to the novel use of anthranilic acid or its derivatives for thinning.

Anthranilic acid is used as an intermediate for production of dyes, pigments and saccharin. It and its esters are also used in preparing perfumes to imitate jasmine and orange, pharmaceuticals (loop diuretics such as furosemide) and UV-absorbers, as well as corrosion inhibitors for metals and mold inhibitors in soya sauce. Its usefulness as a chemical thinner is surprising.

The present invention relates to the use of acetaminophen or its derivatives in combination with auxins, anthranilic acid or other auxin-related compounds for thinning.

Acetaminophen is widely used as an over-the-counter analgesic and antipyretic. It will be appreciated that its efficacy as part of a chemical thinning package is surprising.

### STATEMENTS OF THE INVENTION

The present invention is directed to the treatment of a plant with an effective amount of a composition comprising a compound selected from an auxin, an auxin precursor, an auxin metabolite or a derivative of said auxin, auxin precursor or auxin metabolite or a mixture thereof and acetaminophen or a derivative thereof, at the flowering or fruitlet stage to reduce the final number of fruits that the plant sets and grows to maturity.

The present invention is also directed to the treatment of a plant with an effective amount of anthranilic acid (also referred to as “AN”) or an effective salt, ester, or amide thereof including analogs of AN and effective salts, esters and amides thereof, at the flowering or fruitlet stage to reduce the final number of fruits that the plant sets and grows to maturity.

By analog we include a compound that has a similar structure, i.e. same or similar active moiety, and similar chemical properties, e.g. with AN is capable of effecting chemical thinning.

According to one aspect of the present invention there is provided a composition comprising a compound selected from an auxin, an auxin precursor, an auxin metabolite or a derivative of said auxin, auxin precursor or auxin metabolite or a mixture thereof and acetaminophen or an analog or derivative thereof for use as a chemical thinning agent.

According to another aspect of the present invention there is provided anthranilic acid or an analog or a derivative thereof for use as a chemical thinning agent. In other words the present invention is a composition for chemically thinning flowering or fruiting plants comprising, or consisting essentially of, or consisting of one or more of AN or an analog or a derivative thereof.

In one embodiment the derivative of AN or its analog is a salt, an ester, or an amide of the acid, or a conjugate of any of the foregoing.

In one embodiment the derivative compound used in the present invention is in the form of a conjugate, e.g. conjugated to a sugar, an alcohol, an amino acid, a peptide or a protein.

In one embodiment the analog of AN is a compound having the structure shown in FIG. 1.

For ease of reference we will refer to all of the above mentioned AN, analogs and derivatives thereof as “AN-related compounds”.

The present invention also provides a composition comprising a compound selected from an auxin, an auxin precursor, an auxin metabolite or a derivative of said auxin, auxin precursor or auxin metabolite or a mixture thereof and acetaminophen or an analog or derivative thereof wherein the auxin precursor is not AN.

For ease of reference we will also refer to auxin, an auxin precursor, an auxin metabolite or a derivative of said auxin, auxin precursor or auxin metabolite or derivative of said auxin, auxin precursor or auxin metabolite as an “auxin-related compound”.

In another embodiment there is provided a composition comprising a compound selected from an auxin, an auxin precursor, an auxin metabolite or a derivative of said auxin, auxin precursor or auxin metabolite or a mixture thereof, and a further agrochemically acceptable component for use as a chemical thinning agent and optionally acetaminophen or an analog or derivative thereof.

By “agrochemically acceptable component” we include components that are tolerated by a plant, and ideally which are beneficial to a plant.

In one embodiment the auxin-related compound is based on an indolic ring. In another embodiment the auxin-related compound is based on a phenolic ring.

In one embodiment the derivative is an acid, a conjugate, a salt, an ester, or an amide of the auxin, auxin precursor, or auxin metabolite.

In one embodiment the derivative is in the form of a conjugate, e.g. conjugated to a sugar, an alcohol, an amino acid, a peptide or a protein.

In one embodiment the auxin precursor is chorismate, anthranilic acid, phosphoribosyl anthraniliate, 1-(O-carboxyphenulamino)-1-deoxyribulose-5-phosphate, indole-3-glycerol-phosphate, indole, indole-3-acetic acid, tryptophan, tryptamine, N-hydroxy tryptamine, indole-3-acetaldoxime, 1-aci-nitro-2-indolylethane, indolic glucosinate, indole-3-acetonitrile (IAN), indole-3-acetaldehyde, indole-3-lactic acid, indole-3-pyruvic acid, or indole-3-ethanol.

In a particularly preferred embodiment the auxin precursor is anthranilic acid or a derivative thereof as set out above.

The auxin-related compound may be a natural, such as is obtainable from seaweed or algae, or synthetic auxin.

In one embodiment the natural auxin is indole-3-acetic acid (IAA), 4-chloro-indole-3-acetic acid (4-Cl-IAA), phenylacetic acid (PAA), indole-3-butyric acid (IBA), indole-3-acetyl-1-O-β-D-glucose (IAAglc).

In one embodiment the conjugate of the natural auxin is IAA-Inositol, IAA-Inositol-arabinose, IAP1, an IAA-peptide, an IAA glycoprotein, an IAA-glucan, IAA-aspartate, IAA-glucose, IAA-1-O-glucose, IAA-myo-Inositol, IAA-4-O-glucose, IAA-6-O-glucose, IAA-Inositol-galactose, an IAA amide conjugate, or an IAA-amino acid conjugate.

In one embodiment the synthetic auxin is 1-naphthaleneacetic acid (NAA), 2,4-dichlorophenoxyacetic acid (2,4-D), 2-methoxy-3,6-dichlorobenzoic acid (dicamba), 4-amino-3,5,6-trichloropicolinic acid (tordon), 2,4,5-trichlorophenoxyacetic acid (2,4,5-T), 2,3,6-trichlorobenzoic acid, 4-chloro-2 methylphenoxyacetic acid (MCPA) or N,N-dimethylethylthiocarbamate.

In one embodiment the auxin metabolite is indole-3-lactic acid or indole-3-ethanol.

In one embodiment the acetaminophen derivative is a compound as set out in FIG. 3.

In another aspect of the present invention there is provided a composition for use as a chemical thinning agent comprising a compound or a composition as described above in combination with a further chemical thinning agent.

Examples of such further chemical thinning agents include benzyladenine, 1-naphthylacetic acid, carbaryl, (2-chlorophenoxy)propionic acid, ethephon, naphthaleneacetamide, thidiazuron, ammonium thiosulphate, DNOC, endothallic acid, gibberellic acid, lime sulphur, sulfcarbamide and pelargonic acid.

In one embodiment the further agrochemically acceptable component comprises at least one compound selected from a) glucose, hydrolysed starch, sucrose, fructose, glycerol, glyceraldehydes, erythrose, ribulose, xylulose or arabinose, monosaccharides including aldoses such as D-Ribose, D-Xylose, L-Arabinose, D-Glucose, D-Mannose and D-Galactose; ketoses such as D-Ribulose and D-Fructose; deoxyaldoses such as 2-Deoxy-D-ribose, L-Fuccose; acetylated amino sugars such as N-Acteyl-D-glucosamine and N-Acetyl-D-galactosamine; acidic monosaccharides such as D-Glucuronic acid, L-Iduronic acid and N-Acetylneuraminic acid, Sugar alcohols such as D-Sorbitol and D-Mannitol, disaccharides including maltose, lactose and sucrose, or an ester or glycoside or metabolic equivalent of such a carbohydrate; b) an organic acid of the Krebs tricarboxylic acid cycle or a metabolic precursor thereof; c) a vitamin or coenzyme, or a precursor thereof; d) a purine or pyrimidine nucleoside, nucleotide or metabolic precursor thereof; e) a naturally occurring fat or oil; or f) an amino acid.

Preferably the further agrochemically acceptable component comprises at least one compound selected from c) a vitamin or coenzyme, or a precursor thereof; d) a purine or pyrimidine nucleoside, nucleotide or metabolic precursor thereof; or f) an amino acid.

In one embodiment the AN-related compound or the composition of the present invention is for use as a fruit thinner.

In another embodiment the AN-related compound or the composition of the present invention is for use as a flower thinner.

In another aspect of the present invention there is provided a method of chemical thinning comprising applying a chemically thinning effective amount of the AN-related compound or the composition of the present invention to a plant, or its locus.

Thus the present invention relates to a method for applying to flowering or fruiting plants an effective amount of an AN-related compound alone or in combination with simultaneous or sequential applications of acetaminophen or a derivative thereof and/or another suitable ingredient to reduce the number of flowers and/or reduce the number of fruits that set and mature on the plant.

The present invention also relates to a method for applying to flowering or fruiting plants an effective amount of an auxin-related compound in combination with simultaneous or sequential applications of acetaminophen or a derivative thereof and/or another suitable ingredient to reduce the number of flowers and/or reduce the number of fruits that set and mature on the plant.

Non-limiting examples of such plants include trees, shrubs, vines, vegetables or other crops, or ornamentals.

Non-limiting examples of such fruit include a pome fruit, stone fruit, citrus fruit or kiwi fruit. In one embodiment such non-limiting examples include an apple, pear, plum, cherry, apricot, peach or nectarine tree or a grapevine.

As described below in more detail the components of the present invention may be applied at the same or different times. Thus, we described a kit comprising the components of the present invention at least one of which is in a separate container.

The combination of components according to the present invention may also give rise to a synergistic effect in relation to its thinning effect.

### ADVANTAGES

We have found that an AN-related compound, and particularly AN, when applied alone or in mixture with other agrochemically acceptable compounds is (amongst other benefits in fruit and in flowers) an effective plant bioregulator, wherever this is deemed useful.

We have found that an auxin-related compound, and more particularly an AN-related compound, and even more particularly AN, when applied with acetaminophen as a combination or in mixture with other agrochemically acceptable compounds is (amongst other benefits in fruit or flowers) an effective chemical thinning agent, wherever this is deemed useful.

We have also found that an auxin-related compound, and more particularly an AN-related compound, and even more particularly AN, when applied with acetaminophen in mixture with an additive such as at least one compound selected from a) glucose, hydrolysed starch, sucrose, fructose, glycerol, glyceraldehydes, erythrose, ribulose, xylulose or arabinose, monosaccharides including aldoses such as D-Ribose, D-Xylose, L-Arabinose, D-Glucose, D-Mannose and D-Galactose; ketoses such as D-Ribulose and D-Fructose; deoxyaldoses such as 2-Deoxy-D-ribose, L-Fuccose; acetylated amino sugars such as N-Acteyl-D-glucosamine and N-Acetyl-D-galactosamine; acidic monosaccharides such as D-Glucuronic acid, L-Iduronic acid and N-Acetylneuraminic acid, Sugar alcohols such as D-Sorbitol and D-Mannitol, disaccharides including maltose, lactose and sucrose, or an ester or glycoside or metabolic equivalent of such a carbohydrate; b) an organic acid of the Krebs tricarboxylic acid cycle or a metabolic precursor thereof; c) a vitamin or coenzyme, or a precursor thereof; d) a purine or pyrimidine nucleoside, nucleotide or metabolic precursor thereof; e) a naturally occurring fat or oil; or f) an amino acid is (amongst other benefits in fruit) an effective chemical thinning agent, wherever this is deemed useful.

We have found that the above compounds and compositions act as chemical thinning agents and improve crop safety when added to a range of species. Such compounds or compositions would be helpful either when used on their own, or in combination with other chemical thinning agents on the market, e.g. in order to improve their safety, efficacy or economic attractiveness.

We have found that the present invention can provide less phytotoxicity, and/or no or fewer pygmy fruit compared to some commercial standard chemical thinning agents.

We have also found that the present invention allows a wider application window than commercial standard chemical thinning agents.

We have also found the present invention provides an improvement in at least one or more of: lower fruit number, proportion and size of fruit; reduction in lower quality sized fruit and/or skin quality (low russeting score).

### BRIEF DESCRIPTION OF THE FIGURES

FIG. 1 shows structures of examples of analogs of anthranilic acid.

FIG. 2 shows structures of examples of naturally occurring auxins and conjugates.

FIG. 3 shows structures of examples of derivatives of acetaminophen.

FIG. 4 shows an overview of the reactions leading from chorismate to IAA and tryptophan.

FIG. 5 shows the structure of some synthetic auxins.

FIG. 6 shows the results of the trials described below (TAMPF formulation).

FIG. 7 shows the results of the trials described below (untreated).

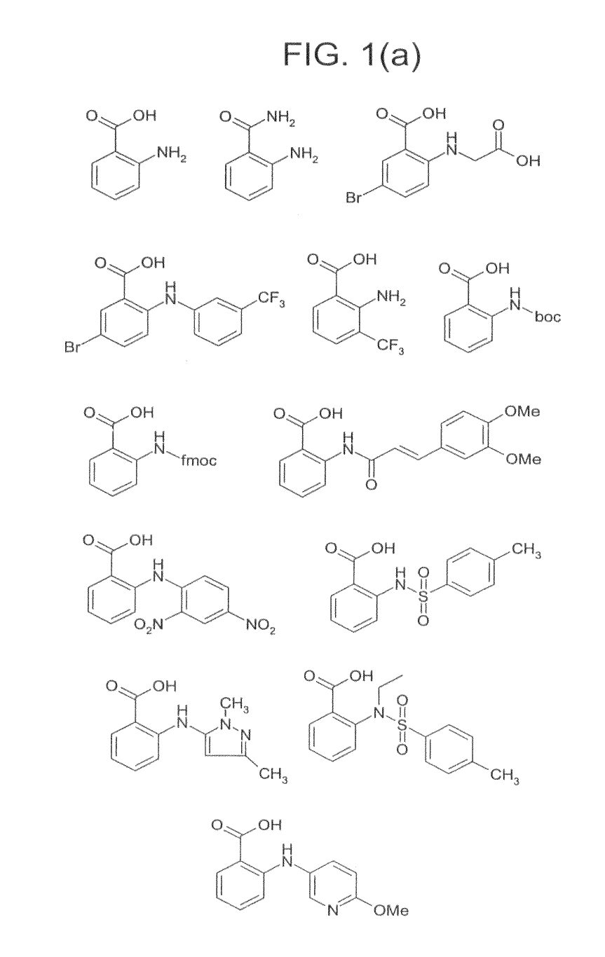

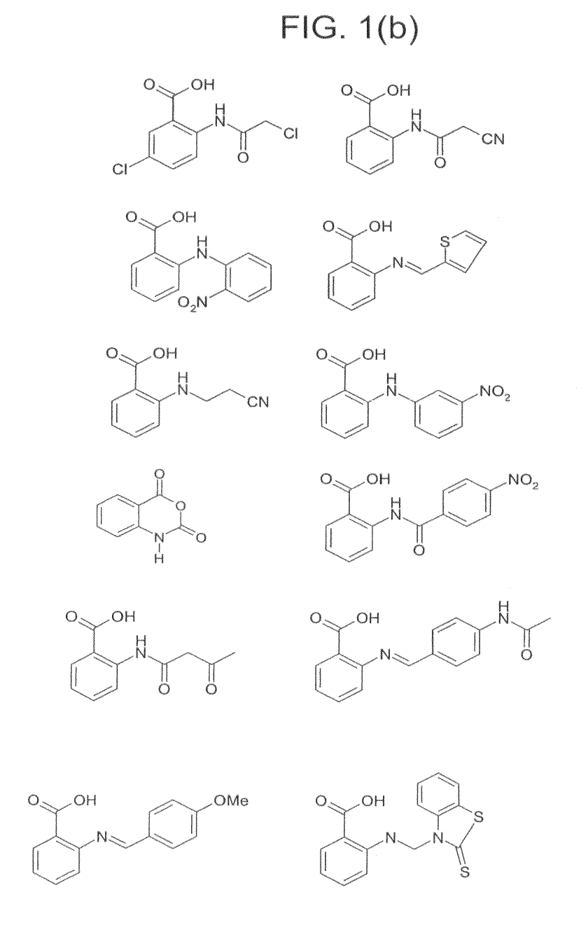

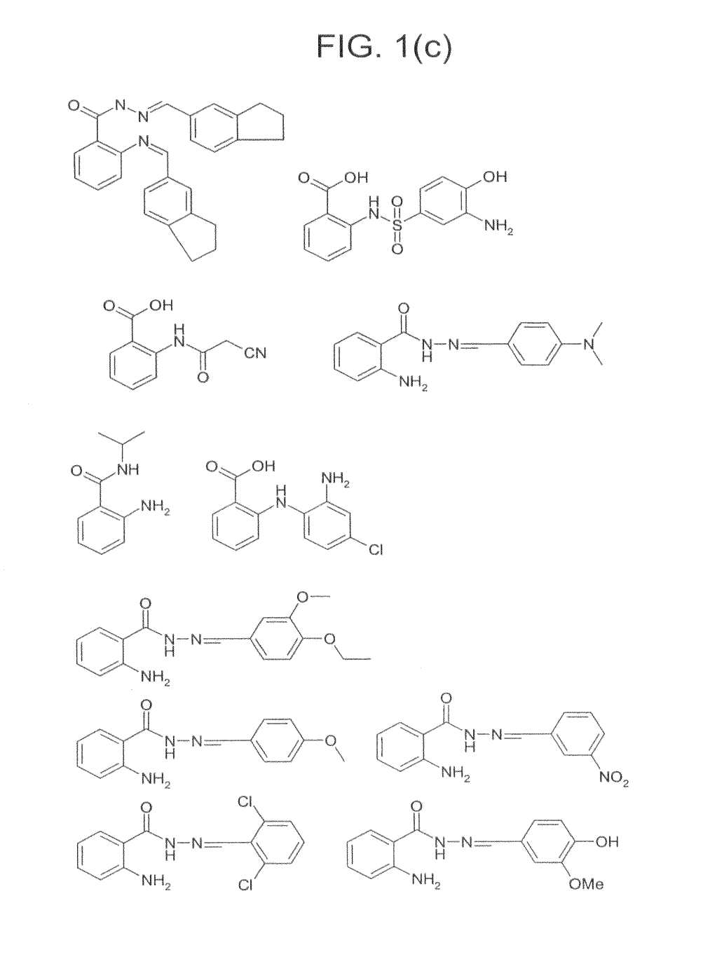

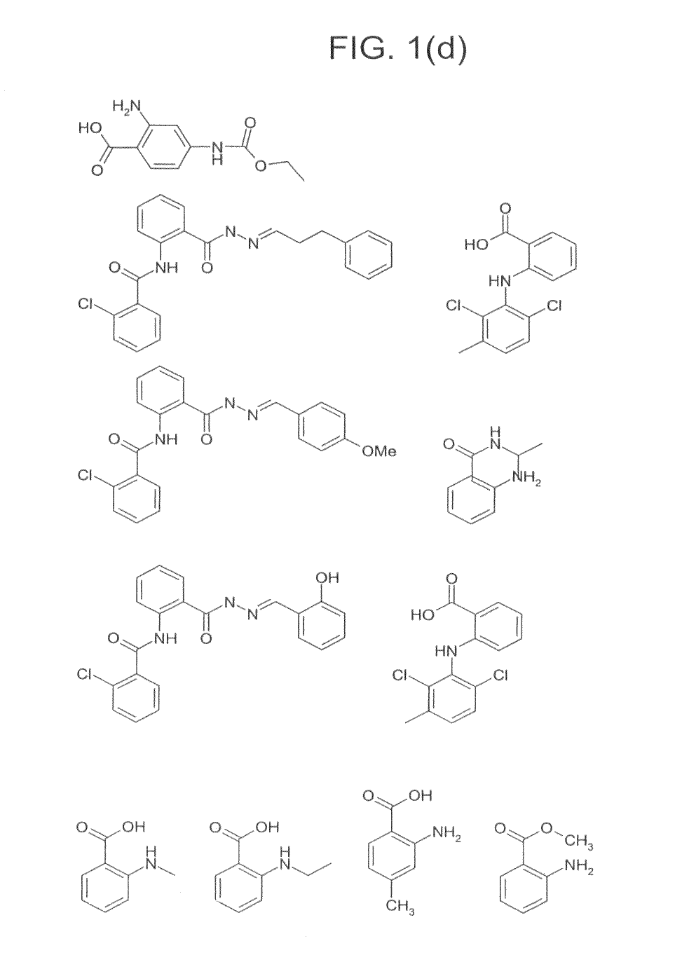

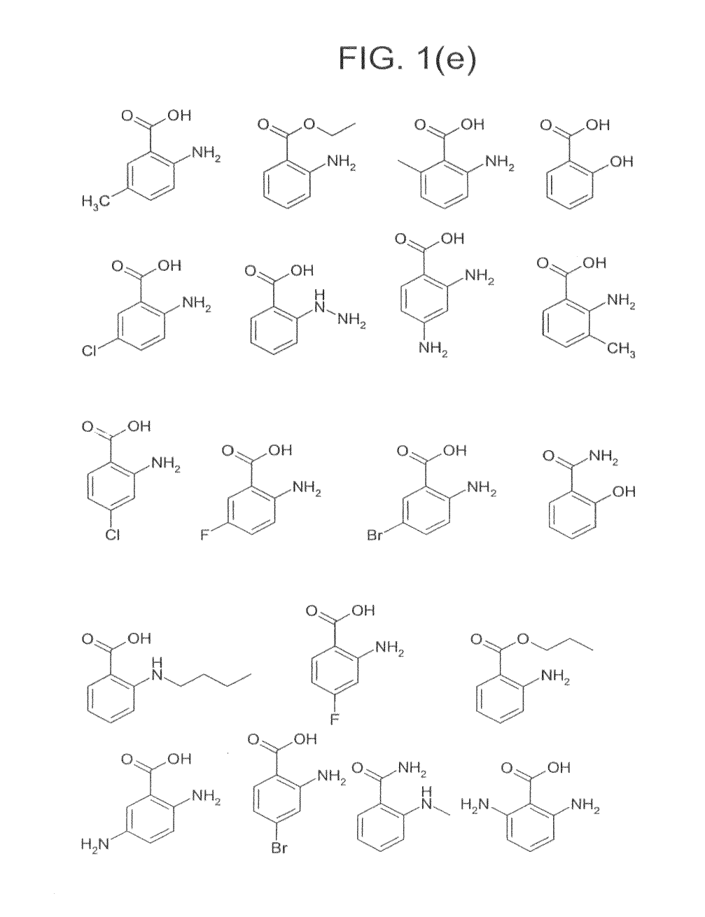

### DETAILED DESCRIPTION OF THE INVENTION

Various preferred features and embodiments of the present invention will now be described by way of non-limiting example.

The invention provides a process and a compound or a composition for thinning fruit blossoms and fruitlets on fruit trees. The process of the invention includes applying an effective amount of a thinning compound or composition to the blossoms and/or fruitlets of a fruit bearing plant.

By “effective amount” we include an amount of the compound or composition of the present invention which is sufficient to achieve the desired “thinning response”. In general by “thinning response” we mean a reduction in amount of fruit and/or flowers present on a plant compared to a control.

The present invention relates to the use of anthranilic acid (AN):

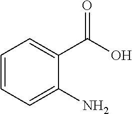

AN, also known as anthraniliate, has the CAS number 118-92-3.

We have described useful derivatives of AN above. Preferably such derivatives are water soluble. Representative salts include inorganic salts such as ammonium, lithium, sodium, potassium, magnesium and calcium salts and organic amine salts such as the triethanolamine, dimethylethanolamine and ethanolamine salts.

The present invention involves the use of auxins.

The fact that auxins can be used as a chemical thinner may be seen as surprising, as some reports indicate that auxins play a role in the initiation of flowering. Auxins have also been used to promote uniform flowering, to promote fruit set and to prevent premature fruit drop. It has also been reported that auxins are required for fruit growth.

Auxins are a class of plant growth hormones. An auxin is an organic substance that promotes cell elongation growth when applied in low concentrations to plant tissue segments in a bioassay. The most studied member of the auxin family is indole-3-acetic acid (IAA). In addition to IAA, there are several other naturally occurring auxins that have been described to date: IAA, IBA, PAA and 4-Cl-IAA. Naturally occurring auxins are found in plants as the free acid and in conjugated forms.

An auxin has been defined as a compound that gives rise to curvature in the grass coleoptile curvature (or growth) test. Such an assay is described by Fritz Went in 1926 and 1928. In this bioassay coleoptile tips of grass seedlings are placed on an agar plate containing the substance to be assayed. If an auxin response is present then the coleoptile bends in darkness and the angle of curvature can be measured. Went's results indicated that the curvatures of stems were proportional to the amount of growth substance in the agar. This test is also called the avena curvature test. Other functional tests which can be employed to determine auxin activity include the ability to cause rooting in stem cuttings and the ability to promote cell division in tissue or cell culture.

A review of auxins, their synthesis and metabolism can be found in e.g. Normanly, Slovin and Cohen in “Plant Hormones, Biosynthesis, Signal Transduction and Action!”, Ed Peter J. Davies, [2004] Chapter “B1. Auxin Biosynthesis and Metabolism” pages 36-62.

In addition to indolic auxins, various phenolic auxins have auxin activity.

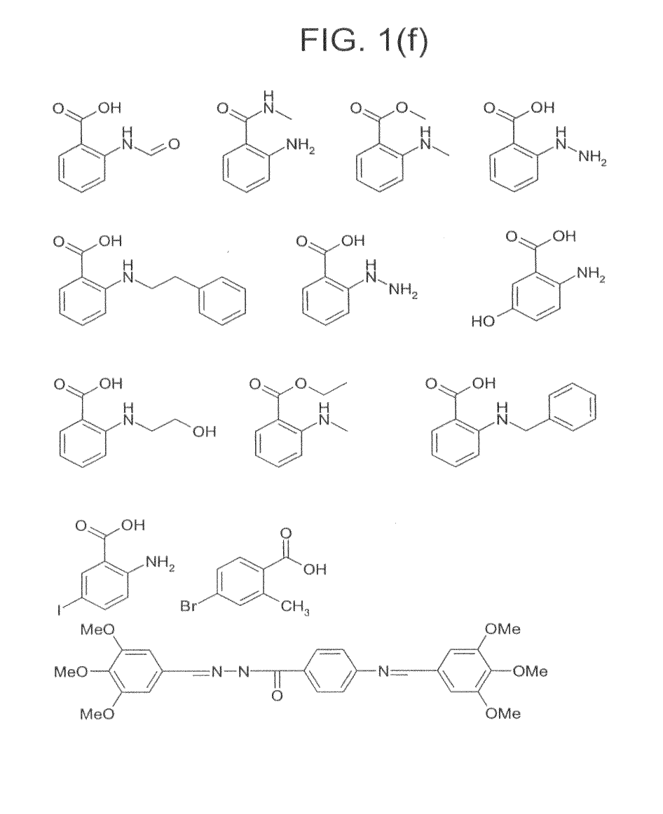

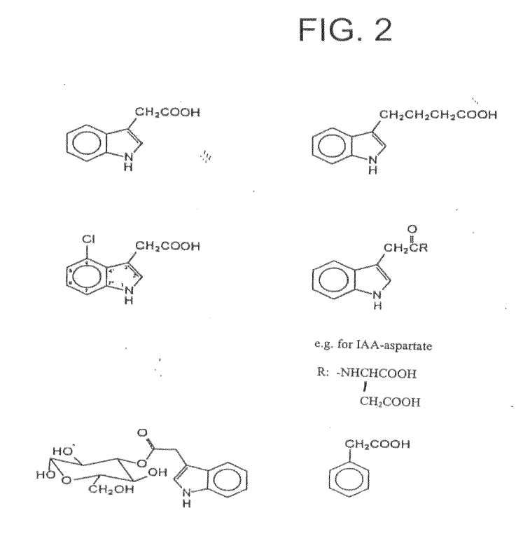

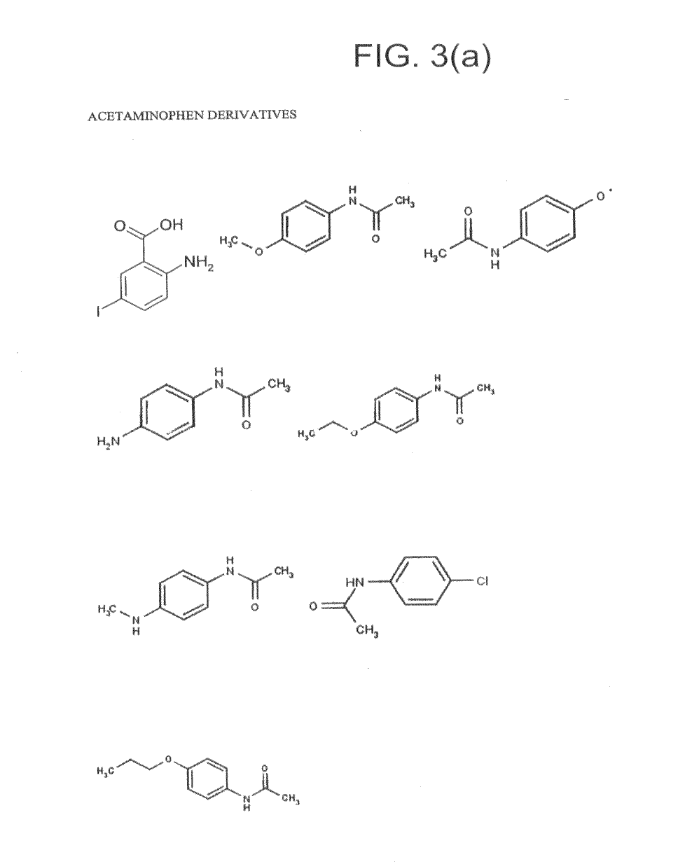

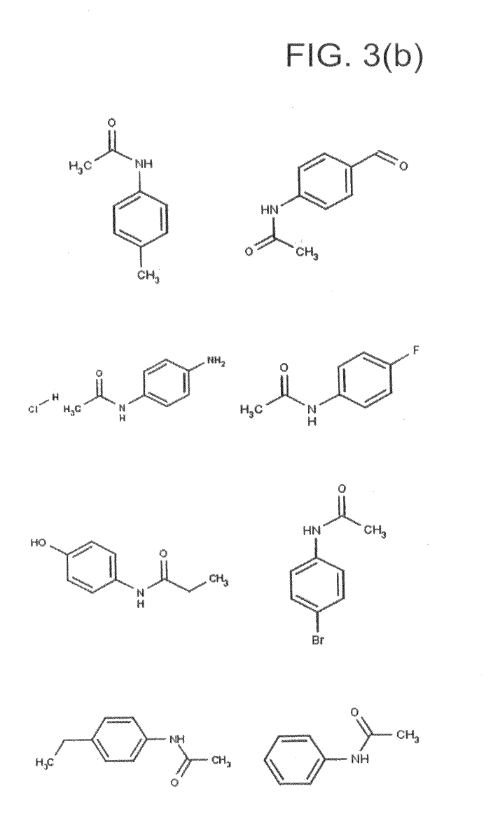

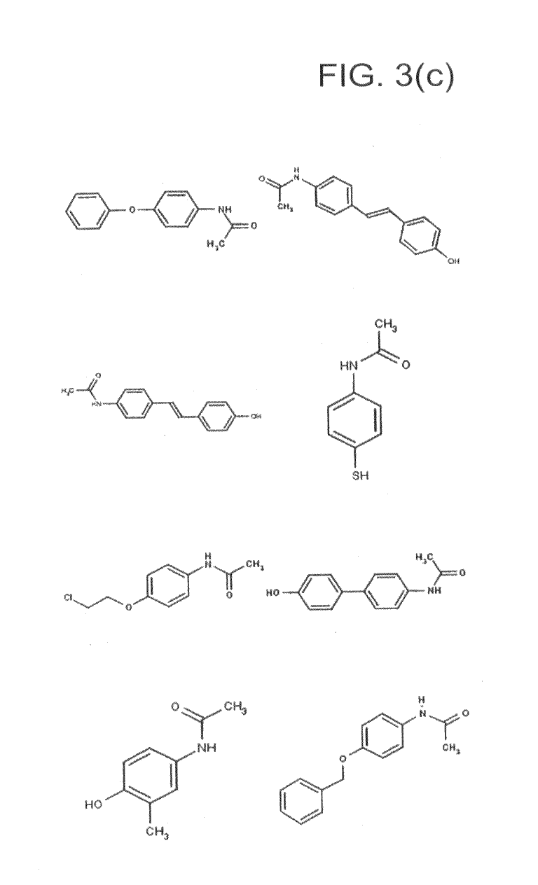

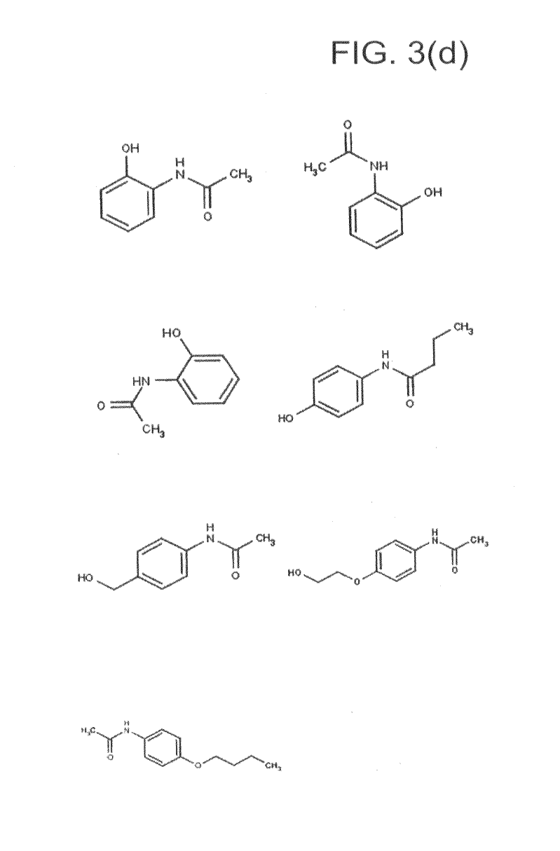

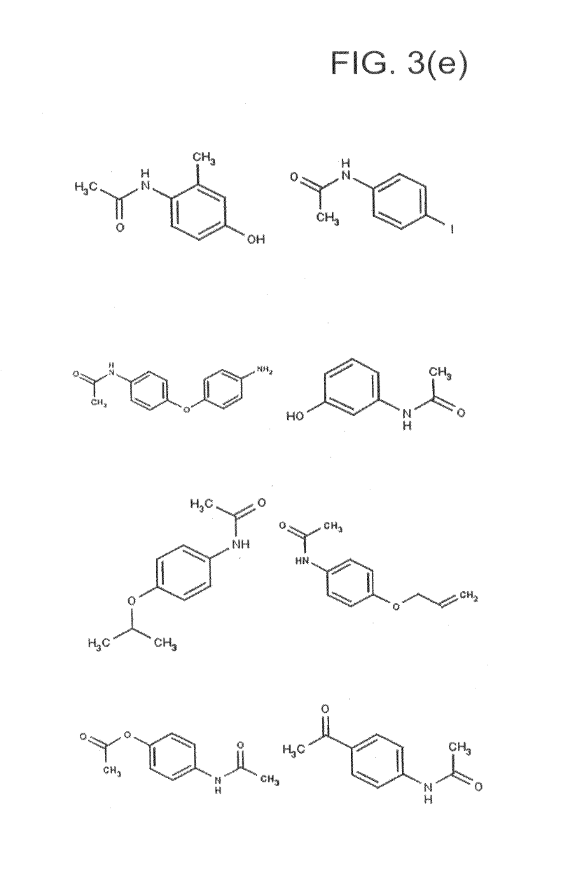

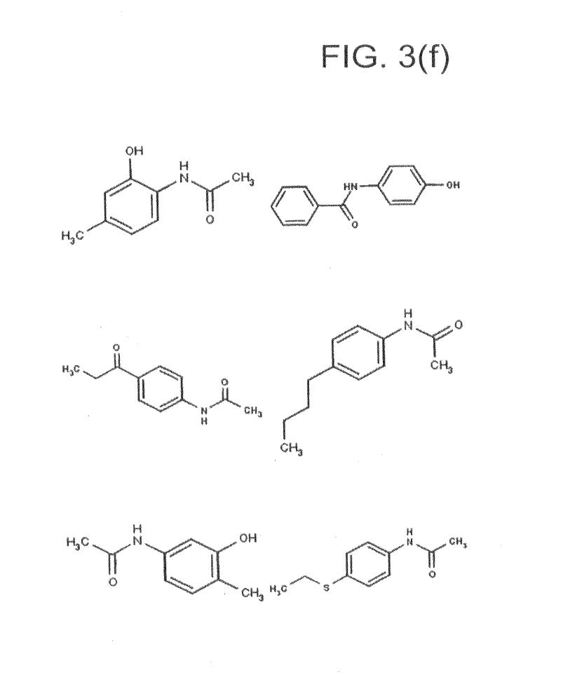

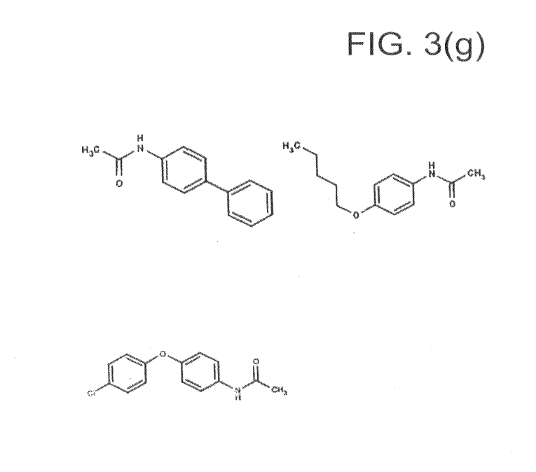

Some examples of naturally occurring auxins and some examples of the lower molecular weight conjugates which may be used in the present invention are shown in FIG. 2.

The present invention may also make use of conjugates. It is believed that plants use conjugates for storage purposes and/or to regulate the amount of free auxin available in the plant. IAA is primarily conjugated to the amino acid aspartate.

Related low molecular weight conjugates, such as IAA-Inos, IAA-Inos-arabinose and conjugates with other amino acids, and higher molecular weight conjugates, such as the IAA protein IAP1, IAA-peptides, IAA glycoprotein and IAA-glucans, have also been isolated from plants.

IAA and its precursors undergo metabolic conversions to indole-3-lactic acid, indole-3-ethanol and IBA. IBA has been found to occur naturally in plants; although some references refer to it as a synthetic auxin. Some commentators refer to it as an auxin per se and others as a precursor to IAA.

One general class of conjugated forms consists of those linked through carbon-oxygen-carbon bridges. These compounds have been referred to generically as “ester-linked”, although some 1-O sugar conjugates such as 1-O-IAA-Gluc are actually linked by acyl alkyl acetal bonds. Typical ester-linked moieties include 6-O-IAGluc, IAA-Inos, IAA-glycoproteins, IAA-glucans and simple methyl and ethyl esters. The other type of conjugates present in plants are linked through carbon-nitrogen-carbon amide bonds (referred to as “amide-linked”), as in the IAA-amino acid and protein and peptide conjugates (see FIG. 2).

Biochemical pathways that result in IAA production within a plant tissue include: (A) de novo synthesis, whether from tryptophan [referred to as Tip-dependent (Trp-D) IAA synthesis], or from indolic precursors of Trp [referred to as Tip-independent (Trp-I) IAA synthesis, since these pathways bypass Trp]; (B) hydrolysis of both amide- and ester-linked IAA conjugates; (C) transport from one site in the plant to another site; and (D) conversion of IBA to IAA. IAA turnover mechanisms include: (E) oxidative catabolism; (F) conjugate synthesis; (G) transport away from a given site; and (H) conversion of IAA to IBA. The present invention makes use of such precursors and metabolites along this pathway. The present invention does not make use of inactive metabolites, such as arise from catabolism of the auxin.

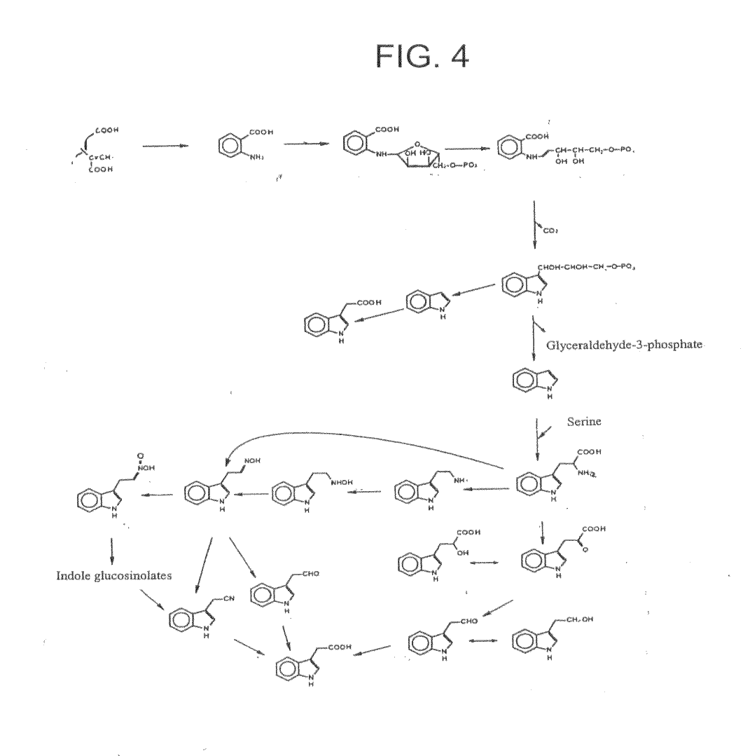

Normally the present invention makes use of the tryptophan-dependent pathway. A summary of the reactions leading from chorismate—the first committed step of indolic metabolism—to IAA and tryptophan is shown in FIG. 4.

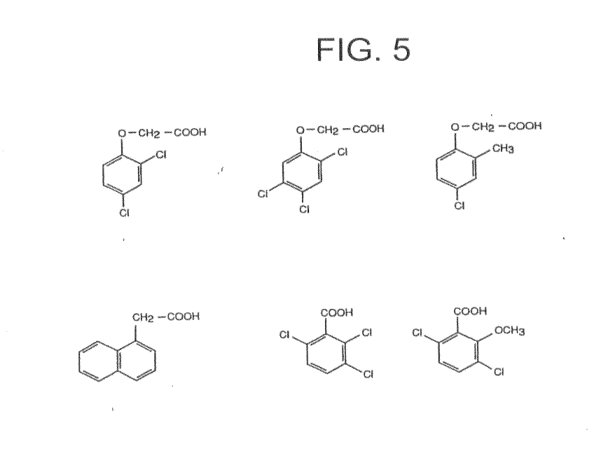

The present invention also encompasses the use of synthetic auxins. Some examples of synthetic auxins are shown in FIG. 5.

A comparison of the compounds that possess auxin activity reveals that at neutral pH they all have a strong negative charge on the carboxyl group of the side chain that is separated from a weaker positive charge on the ring structure by a distance of about 0.5 nm. It has been proposed that an indole is not essential for activity, but that it can be an aromatic or fused aromatic ring of a similar size. A model has been proposed as being a planar aromatic ring-binding platform, a carboxylic acid-binding site and a hydrophobic transition region that separates the two binding sites.

The present invention involves the use of acetaminophen in some embodiments.

Acetaminophen has the IUPAC name, N-(4-hydroxypheyl)acetamide and is commonly referred to as paracetamol. It has the CAS number 103-90-2.

Its formula is:

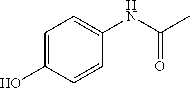

As described above, derivatives of acetaminophen are also useful in the present invention.

The compounds or compositions of the present invention can be used in combination with other components, as appropriate.

In one preferred embodiment one such component may be an additive as defined as belonging to one or more of the following classes (a) to (f); although two or more such additives in the same or different classes may be used:
(a) glucose, hydrolysed starch, fructose, glycerol, glyceraldehyde, erythrose, xylulose or arabinose, monosaccharides including aldoses such as D-Ribose, D-Xylose, L-Arabinose, D-Glucose, D-Mannose and D-Galactose; ketoses such as D-Ribulose and D-Fructose; deoxyaldoses such as 2-Deoxy-D-ribose, L-Fuccose; acetylated amino sugars such as N-Acteyl-D-glucosamine and N-Acetyl-D-galactosamine; acidic monosaccharides such as D-Glucuronic acid, L-Iduronic acid and N-Acetylneuraminic acid, Sugar alcohols such as D-Sorbitol and D-Mannitol, disaccharides including maltose, lactose and sucrose, or an ester or glycoside or metabolic equivalent of such a carbohydrate, which will normally be applied at 10 to 10,000 g/ha (grams per hectare). Without wishing to be bound by any theory the component may function as
(1) A source for the production of high energy bonds as in adenosine trisephosphate (ATP) production,
(2) For the formation of reduced nicotinamide adenine dinucleotide (NADH) and reduced nicotamide adenine dinucleotide phosphate (NADPH) and
(3) As precursors of amino acids and nucleotides;
(b) an organic acid of the Krebs Tricarboxylic Acid Cycle or a metabolic precursor thereof, (including citric, succinic, malic, pyruvic, acetic and fumaric acids), which will normally be applied at similar rates to and used for similar functions as the carbohydrate source;
(c) a vitamin or coenzyme, e.g. thiamine, riboflavin, pyridozine, pyridoxamine, pyridoxal, nicotinamide, folic acid, or a precursor thereof including nicotinic acid, which will normally be applied at 0.01 to 500 g/ha to stimulate metabolic processes dependent on enzymatic action;
(d) a purine or pyrimidine nucleoside, nucleotide or a metabolic precursor thereof, e.g. adenine, adenosine, thymine, thymidine, cytosine, guanine, guanosine, hypoxanthine, uracil, uridine or inosine, which will normally be applied at 1 to 500 g/ha to act as structural precursors for nucleic acid synthesis;
(e) a naturally occurring fat or oil including olive, soya, coconut and corn oils, which can be degraded by living organisms to fatty acids and which will normally be applied at 10 to 10,000 g/ha;
(f) an amino acid of a type that occurs naturally in plant proteins, e.g. glycine, alanine, valine, leucine, isoleucine, serine, threonine, cysteine, methionine, aspartic acid, glutamic, acid, glutamine, asparagine, lysine, hyroxylysine, arginine, histidine, phenylalanine, tyrosine, tryptophan, proline or hydroxyproline, which will normally be applied at 1 to 500 g/ha to act as structural units for newly formed proteins or by their degradation to function in a similar manner to fatty acids and carbohydrates.

Other ingredients such as adjuvants may be added to the thinning solution. The adjuvants can facilitate spreading and efficacy, and improve the adhesion properties of the composition, and generally include oils, antifoaming agents and surfactants. Such components which are useful in the present invention include, but are not limited to: terpene, Brij family (polyoxyethylene fatty alcohol ether) from Uniqema (Castle, Del.); surfactant in Tween family (Polyoxyethylene sorbitan esters) from Uniqema (Castle, Del.); Silwet family (Organosilicone) from Union Carbide (Lisle, Ill.); Triton family (Octylphenol ethoxylate) from The Dow Chemical Company (Midland, Mich.); Tomadol family (ethoxylated linear alcohol) from Tomah3 Products, Inc. (Milton, Wis.); Myrj family (Polyoxyethylene (POE) fatty acid esters) from Uniqema (Castle, Del.); Span family (Sorbitan ester) from Uniqema (Castle, Del.); and Trylox family (Ethoxylated Sorbitol and Ethoxylated Sorbitol Esters) from Cognis Corporation (Cincinnati, Ohio) as well as commercial surfactant Latron B-1956 (77.0% modified phthalic/glycerol alkyl resin and 23.0% Butyl alcohol) from Rohm & Haas (Philadelphia, Pa.); Caspil (Blend of Polyether-polymethylsiloxanecopolymer and nonionic surfactant) from Aquatrols (Paulsboro, N.J.); Agral 90 (Nonyl phenol ethoxylate) from Norac Concept, Inc. (Orleans, Ontario, Canada); Kinetic (99.00% Proprietary blend of polyalkyleneoxide modified polydimethylsiloxane and nonionic surfactants) from Setre Chemical Company (Memphis, Tenn.); and Regulaid (90.6% 2-butoxyethanol, poloxalene, monopropylene glycol) from KALO, Inc. (Overland Park, Kans.).

When the final solution is to be applied to plants which, because of their hairy or waxy surface, may be difficult to wet, it may be particularly advantageous to include such other additives, commonly known in the agrochemical industry, such as surfactants, wetting agents, spreaders and stickers. (Examples of wetting agents include silicone surfactants, nonionic surfactants such as alkyl ethoxylates, anionic surfactants such as phosphate ester salts and amphoteric or cationic surfactants such as fatty acid amido alkyl betaines).

The compounds or compositions of the invention may be the sole active ingredient of the composition or they may be admixed with one or more additional active ingredients such as nematicides, insecticides, synergists, herbicides, fungicides, fertilisers or plant growth regulators where appropriate.

The compounds or compositions of the present invention can also be used in combination with other chemical thinning agents, such as benzyladenine, 1-naphthylacetic acid, carbaryl, (2-chlorophenoxy) propionic acid, ethephon, napthaleneacetamide, thidiazuron, ammonium thiosulphate, DNOC, endothallic acid, ethephon, gibberellic acid, lime sulphur, sulfcarbamide, pelargonic acid, 6-benzylaminopurine, N-(2-chloro-4-pyridyl)-N-phenylurea, and thidiazuron.

In a particularly preferred embodiment, the one or more compounds of the invention are administered in combination optionally with one or more active agents. In such cases, the compounds of the invention may be administered consecutively, simultaneously or sequentially with each other or the one or more active agents. The major advantages of combining the compounds are that it may promote additive or possible synergistic effects through e.g. biochemical interactions. Beneficial combinations may be suggested by studying the activity of the test compounds. This procedure can also be used to determine the order of administration of the agents, i.e. before, simultaneously or after delivery.

Chemical thinning is the regulation of crop load through the addition of a compound that reduces flowering and crop load in established orchards. The chemical thinners of the present invention can have two major effects: (1) increased fruit size and quality and (2) maintenance of annual bearing, i.e. enhanced return bloom.

In pome fruit, chemical thinners are commonly applied between full bloom and the cessation of cell division, which occurs about a month after full bloom. The exact timing of thinner application depends on material chosen, cultivar, and local conditions, e.g. in stone fruit application may occur prebloom.

Chemical thinning is generally performed by fruit growers each season. The degree of thinning and the effect on return bloom the following season depends on a number of factors: variety and strain, tree condition, fruit set, proximity to pollinizers, weather, the chemical, and application method.

In order to apply the composition to the plant or environs of the plant, the composition may be used as a concentrate or more usually is formulated into a composition which includes an effective amount of the composition of the present invention together with a suitable inert diluent, carrier material and/or surface active agent. Preferably the composition is in the form of an aqueous solution which may be prepared from the concentrate. By effective amount we mean that the composition (and/or its individual components) provides a chemical thinning effect. The amount of water used per ha would normally be high and the plants would be treated until runoff. The high amount of water per ha is normally necessary for the optimal uptake of the compound by the leaves and the blossoms and fruitlets.

For tree fruit bloom applications, the chemical thinner is applied in a formulation that is preferably a substantially aqueous solution. The chemical thinner solution can be mixed on site in the spray tank or delivered and stored in aqueous solution, to ensure proper mixing and dilution, as appropriate.

The applied amount of chemical thinner can vary widely depending on the water volume applied to plants as well as other factors such as plant age and size, and plant sensitivity to the chemical thinner. Typical rates of AN-related compounds would be 1-100 g/ha (preferably and in these trials, 1 g active ingredient per hectare (of standard orchard was applied)), typical rates of acetaminophen or its derivatives would be 3-300 g/ha (preferably and in these trials, 3 g active ingredient per hectare was applied). Typical rates of agrochemically acceptable additive of the present invention would be 1-10 g/ha (preferably and in these trials, less than 3 g active ingredient per hectare was applied). The rate of other components such as spreaders and stickers can be 50-200 ml per ha.

The rate and timing of application will depend on a number of factors known to those skilled in the art, such as the type of species etc. A second or further application(s) can be made as appropriate. The timings between each application may be in the region of 5 days or more.

The present invention relates to a method of controlling fruit and/or blossom production which comprises applying to the plants or to the locus thereof an effective controlling amount of the compound/compositions of the present invention.

The compositions of the present invention can be applied to the soil, plant, seed, or other area to be protected. Preferably the present invention is applied to the foliage of plants. The composition may be applied in the form of dusting powders, wettable powders, granules (slow or fast release), water dispersible granules, emulsion or suspension concentrates, liquid solutions, emulsions, seed dressings, or controlled release formulations such as microencapsulated granules or suspensions, soil drench, irrigation component, or preferably a foliar spray.

Dusting powders are formulated by mixing the active ingredient with one or more finely divided solid carriers and/or diluents, for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers.

Granules are formed either by absorbing the active ingredient in a porous granular material for example pumice, attapulgite clays, fuller's earth, kieselguhr, diatomaceous earths, ground corn cobs, and the like, or on to hard core materials such as sands, silicates, mineral carbonates, sulfates, phosphates, or the like. Agents which are commonly used to aid in impregnation, binding or coating the solid carriers include aliphatic and aromatic petroleum solvents, alcohols, polyvinyl acetates, polyvinyl alcohols, ethers, ketones, esters, dextrins, sugars and vegetable oils, with the active ingredient. Other additives may also be included, such as emulsifying agents, wetting agents or dispersing agents.

Microencapsulated formulations (microcapsule suspensions CS) or other controlled release formulations may also be used, particularly for slow release over a period of time, and for seed treatment.

Alternatively and preferred the compositions may be in the form of liquid preparations to be used as dips, irrigation additives or sprays, which are generally aqueous dispersions or emulsions of the active ingredient in the presence of one or more known wetting agents, dispersing agents or emulsifying agents (surface active agents). The compositions which are to be used in the form of aqueous dispersions or emulsions are generally supplied in the form of an emulsifiable concentrate (EC) or a suspension concentrate (SC) containing a high proportion of the active ingredient or ingredients. An EC is an homogeneous liquid composition, usually containing the active ingredient dissolved in a substantially non-volatile organic solvent. An SC is a fine particle size dispersion of solid active ingredient in water. To apply the concentrates they are diluted in water and are usually applied by means of a spray to the area to be treated.

Suitable liquid solvents for ECs include methyl ketone, methyl isobutyl ketone, cyclohexanone, xylenes, toluene, chlorobenzene, paraffins, kerosene, white oil, alcohols (for example, butanol), methylnaphthalene, trimethylbenzene, trichloroethylene, N-methyl-2-pyrrolidone and tetrahydrofurfuryl alcohol (THFA).

These concentrates are often required to withstand storage for prolonged periods and after such storage, to be capable of dilution with water to form aqueous preparations which remain homogeneous for a sufficient time to enable them to be applied by conventional spray equipment. The concentrates may contain 1-85% by weight of the active ingredient or ingredients. When diluted to form aqueous preparations such preparations may contain varying amounts of the active ingredient depending upon the purpose for which they are to be used.

The composition may also be formulated as powders (dry seed treatment DS or water dispersible powder WS) or liquids (flowable concentrate FS, liquid seed treatment LS), or microcapsule suspensions CS for use in seed treatments. The formulations can be applied to the seed by standard techniques and through conventional seed treaters. In use the compositions are applied to the plants, to the locus of the plants, by any of the known means of applying fertiliser compositions, for example, by dusting, spraying, or incorporation of granules.

As indicated above, the fertilisers produced according to this present invention are usually applied to the foliage of plants but may also be applied to the soil or added to the irrigation water.

The present invention is useful in relation to fruit crops. The crops can include trees, bushes, shrubs, vines, vegetables or other crops, or ornamentals.

The present invention can be used on the following plants as non-limiting examples: Almond (Prunus dulcis), Apple (Malus domestica), Apricot (Prunus armeniaca), Avocado (Persea americana), Banana, Plantain (Musa spp.), Blackberries (Rubus spp), Blueberries (Vaccinium spp), Cacao or cocoa (Theobroma cacao), Cashew (Anacardium occidentale), Cherries (Prunus cerasus, P. avium), Chestnuts (Castanea spp.), Coconut (Cocos nucifera), Coffee (Coffea arabica, C. canephora), Cranberry (Vaccinium macrocarpon), Currants (Ribes spp), Date (Phoenix dactylifera), Fig (Ficus carica), Gooseberry (Ribes grossularia; R. hirtellum), Grapefruit (Citrus paradisi), Grapes (Vitis vinifera, other Vitis spp), Guava (Psidium guajava & related spp), Hazelnut or filbert (Corylus avellana), Juneberry (Amelanchier alnifolia), Kiwifruit (Actinidia deliciosa), Kumquat (Fortunella spp), Lemon (Citrus limon), Lime (Citrus aurantifolia), Loquat (Eriobotrya japonica), Macadamia (Macadamia integrifolia), Mango (Mangifera indica), Mayhaw (Crataegus spp.), Oil Palm (Elaeis guineensis), Olive (Olea europaea), Orange (Citrus sinensis), Papaya (Carica papaya), Peach (Prunus persica), Pears (Pyrus communis, P. pyrifolia), Pecan (Carya illinoensis), Pineapple (Ananas comosus), Pistachio (Pistacia vera), Plums (Prunus domestica, P. salicina), Pomegranate (Punica granatum), Quince (Cydonia oblonga), Raspberries (Rubus idaeus, R. occidentalis), Strawberry (Fragaria X ananassa), Tangerine (Citrus reticulata), Walnut (Juglans regia), Chrysanthemum, and Rhododendron including Azalea species (e.g. Azaleastrum), Kalanchoe, Bulb crops, Crocus, Tulip, Narcissus, Hyacinth, Poinsettia and Roses.

The following mixtures of the compound or composition of the present invention are particularly mentioned:

1. The addition of Anthranilic Acid (AN).
2. The addition of Acetaminophen (AC) to Anthranilic Acid (AN).
3. The addition of an additive (ADD) to Anthranilic Acid.
4. The addition of ADD to AN+AC.
5. The addition of ADD to NAA/other auxins, or auxin mixes (eg NAA/BA).
6. The addition of ADD to AC+AN/NAA/other auxins, or auxin mixes (eg NAA/BA).
7. The addition of AC to NAA/other auxins or auxin mixes.

These and other combinations in accordance with the present invention may give rise to an additive or synergistic effect.

The additive may be one set out as classes (a) to (f) above.

When the additive is selected from class (a) it is preferably one or more of glucose, sucrose, fructose or glycerol.

When the additive is selected from class (b) it is preferably one or more of citric or succinic acid.

When the additive is selected from class (c) it is preferably one or more of thiamine, riboflavin, pyridoxine, nicotinamide, folic acid, ascorbic acid, biotin or vitamin B12.

When the additive is selected from class (d) it is preferably adenine, thymidine, cytosine or uracil.

When the additive is selected from class (e) it is preferably a corn oil.

When the additive is selected from an amino acid is it preferably one of more of glycine, alanine, valine, leucine, threonine, cysteine, methionine, glutamine, asparagine or lysine.

The following Examples further illustrate, but do not limit, the invention.

### EXPERIMENTAL RESULTS

### Example 1

### Apple—cv Elstar

The TAMPF formulation example in these experiments included AN+AC+ADD, applied at the rate of 1 litre per hectare. ADD=at least one from class (f) each at <3 g/l, plus at least one from class (c).

A. APPLE, cv ELSTAR. Sprayed (single application) 11 June, when mean fruit diameter was 20 mm. Counts undertaken in July.

Number of Fruits Per Branch (average from two branches per tree)
T = Tree, B = Branch (ie representative positions for counts).

| | | | | | | | Replicate | Treatment |
|---|---|---|---|---|---|---|---|---|
| Treatment | T1B2 | T2B2 | T1B3 | T2B3 | T1B4 | T2B4 | Mean | Mean |
| Untreated | | | | | | | | |
| Rep 1 | 3 | 4 | 4 | 2 | 3 | 1 | 2.83 | 3.55 |
| Rep 2 | 3 | 5 | 3 | 4 | 7 | 2 | 4.00 | |
| Rep 3 | 3 | 11 | 3 | 2 | 3 | 1 | 3.83 | |
| Handthinning | | | | | | | | |
| Rep 1 | 2 | 2 | 1 | 2 | 2 | 2 | 1.83 | 2.16 |
| Rep 2 | 2 | 2 | 2 | 1 | 3 | 1 | 1.83 | |
| Rep 3 | 3 | 1 | 2 | 4 | 3 | 4 | 2.83 | |
| TAMPF | | | | | | | | |
| Formulation | | | | | | | | |
| Rep 1 | 1 | 2 | 2 | 2 | 5 | 2 | 2.33 | 2.44 |
| Rep 2 | 3 | 3 | 2 | 1 | 2 | 6 | 2.83 | |
| Rep 3 | 3 | 3 | 2 | 1 | 3 | 1 | 2.17 | |

The results show effective fruit thinning from the TAMPF formulation.

B. APPLE cv Elstar—follow-through results.

| | | | Fruit | | | |
|---|---|---|---|---|---|---|
| | | | Skin | | | |
| | | | Quality: | | | |
| | | Fruit | Fruit | | | |
| | | Set: | Skin | | | |
| | Trunk | No of | Russeting | | Number | Mean |
| | x-Sectional | Fruits | (FSR): | Total | of Fruits | Fruit |
| | Area (cm3) | per | TH4- | Yield | per | Weight |
| | per Tree | Branch | Value: | (Kg/Tree) | Tree: | (g) at |
| | (TCSA) | 16/07 - | 19/08 - | 10/9 - | 10/9 - | Harvest: |
| Treatment | 10/07 - Mean | Mean | Mean | Mean | Mean | 10/9 |
| Untreated | 18.54 | 3.39 | 0.25 | 15.35 | 104.2 | 148.7 |
| Handthinning | 17.84 | 2.45 | 0.13 | 12.33 | 74.8 | 165.1 |
| TAMPF | 22.70 | 2.92 | 0.13 | 13.83 | 87.4 | 159.4 |
| Formulation | | | | | | |

The TAMPF formulation showed fewer fruits set per branch, improved skin quality (reduced russeting), fewer fruits per tree at harvest and greater individual mean fruit weight (g) at harvest, relative to untreated. Good thinning occurred, which resulted in improved weight per fruit.

C. APPLE cv Elstar—follow-through results.

| | % Fruit | | | |
|---|---|---|---|---|
| | Weight | | | |
| | In Fruit Size | % Fruit | % Fruit | % Fruit |
| | Class | Weight | Weight | Weight |
| | <70 mm | In Fruit Size | In Fruit | In Fruit |
| | (lower | Class | Size Class | Size Class |
| Treatment | quality) | 70-80 mm | >80 mm | >70 mm |
| Untreated | 24.49 | 65.32 | 10.20 | 75.52 |
| Handthinning | 10.58 | 70.65 | 18.77 | 89.42 |
| TAMPF | 16.34 | 68.45 | 15.20 | 83.65 |
| Formulation | | | | |

The TAMPF formulation produced a lower percentage of fruit in the lower quality category, and an increased percentage of fruit in the higher quality categories. Thus a greater proportion of larger apples of enhanced market value were produced.

D. APPLE cv Elstar—follow-through results.

| | No of Fruits | | | |
|---|---|---|---|---|
| | In Fruit Size | | No of | No of |
| | Class | No of Fruits | Fruits | Fruits |
| | <70 mm | In Fruit Size | In Fruit | In Fruit |
| | (lower | Class | Size Class | Size Class |
| Treatment | quality) | 70-80 mm | >80 mm | >70 mm |
| Untreated | 33.38 | 63.56 | 7.29 | 70.85 |
| Handthinning | 10.58 | 53.04 | 11.21 | 64.25 |
| TAMPF | 19.69 | 58.31 | 9.44 | 67.75 |
| Formulation | | | | |

The TAMPF formulation produced fewer fruits in the lower quality category, and similar fruit numbers in the higher categories combined (ie >70 mm).

E. APPLE cv Elstar—follow-through results

| | Mean Fruit | | |
|---|---|---|---|
| | Weight (g) | | |
| | in Class | Mean Fruit | Mean Fruit |
| | <70 mm | Weight (g) | Weight (g) |
| | (lower | in Class | in Class |
| Treatment | quality) | 70-80 mm | >80 mm |
| Untreated | 116.6 | 156.9 | 209.0 |
| Handthinning | 123.7 | 163.5 | 209.5 |
| TAMPF | 120.1 | 162.7 | 214.6 |
| Formulation | | | |

The TAMPF formulation produced greater individual fruit weights (g) in each size category, as expected from an effective fruit thinning agent.

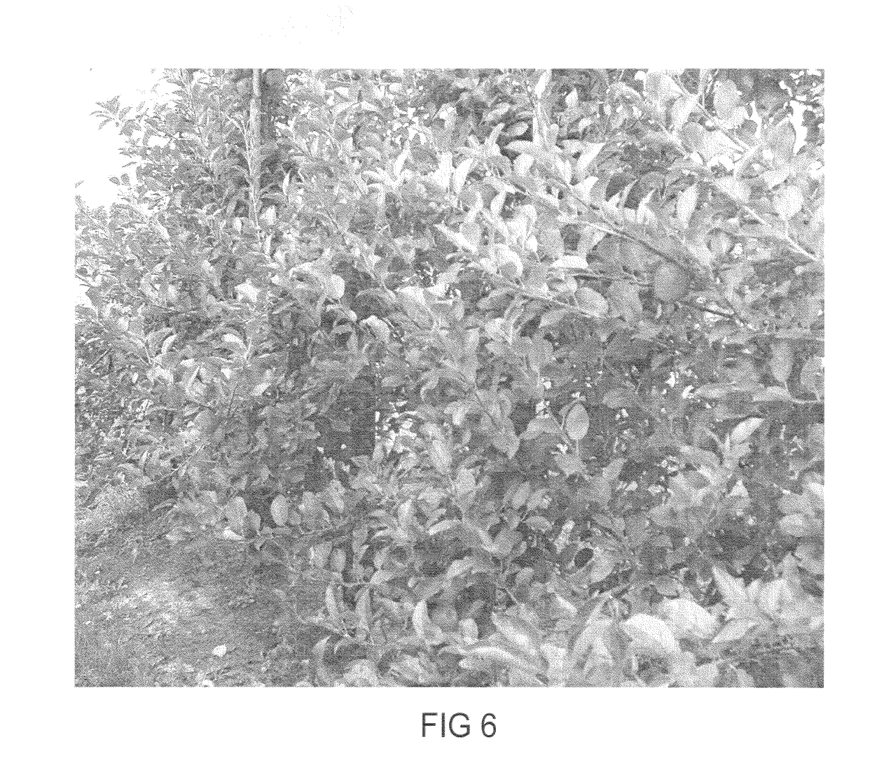

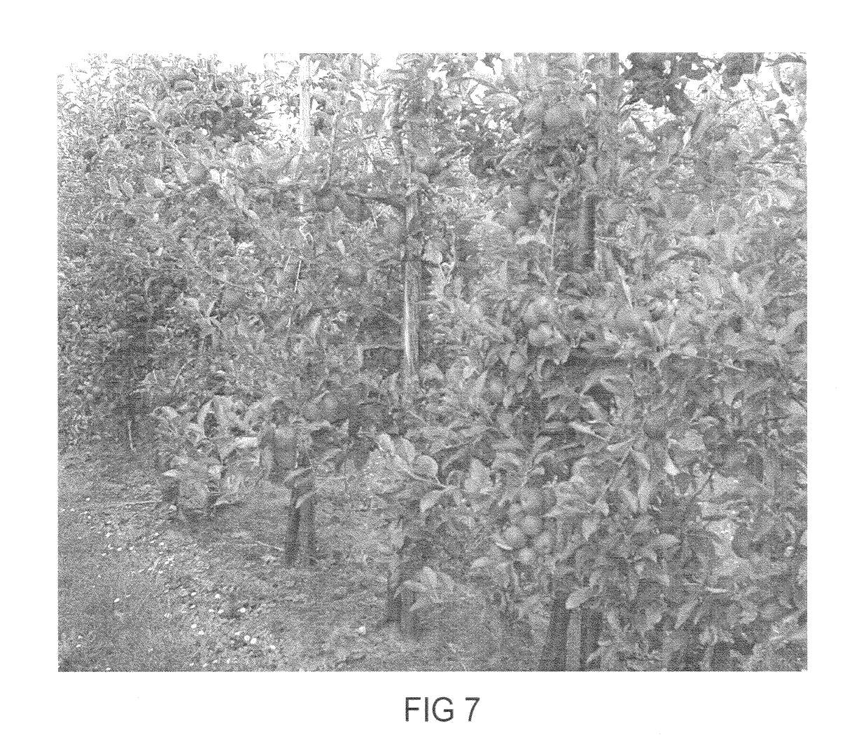

FIG. 6 and FIG. 7 show results from the above trials. The apple trees in FIG. 6 were treated with the TAMPF formulation and the apple trees in FIG. 7 were untreated. The effectiveness of the TAMPF formulation as a fruit thinning agent is evident, in particular, from the smaller number of fruits per branch on the trees treated with the TAMPF formulation.

The TAMPF formulation example in this experiment was applied at the rate of 1 litre per hectare. This proved to be very safe, with no phytotoxicity or any damage whatsoever, and no “pygmy fruit” as is common eg with NAA. Further, application at 20 mm fruit diameter stage was far later than for other fruit thinners, eg NAA, or NAA+Benzyladenine, are not recommended beyond 8-12 mm fruit diameter. This therefore represents an improvement in application window over NAA alone or in mixture. Both average fruit sizes and weights (marketing quality), and skin coloration quality (russeting index) were greatly improved by the TAMPF formulation.

### Example 2

### Chrysanthemum—cv Herby Mixed

A. To demonstrate the benefits to flower bud thinning of applying an auxin/auxin precursor or auxin/auxin precursor plus acetaminophen.

Cultural Details: plants were sown in a commercial seed compost and then “pricked out” into a professional grade all-purpose compost in 9 cm pots set out in a randomized block design. Normal daylight plus supplementary lighting to provide a minimum of 10 hours per day. Heating was given to a minimum of 60 degrees Fahrenheit during the day and 40 degrees F. at night.

Sowing Date: 5 Oct. 2009, in plugs. Application Date: 2 Dec. 2009 (first bud). Measurement Date: 12 Jan. 2010 (flowering). Measurement: Number of buds per plant.

A number of auxins or auxin precursors were applied, each at 10⁻² Molar solutions. Addition of acetaminophen (AC) was at 3 g per hectare equivalent in each test.

| | | % | |
|---|---|---|---|
| | | Improvement | |
| | | from | % |
| | | addition of | Improvement |
| | Mean | AC to auxin/ | over control |
| | Number | precursor. | ie (C) |
| | of Buds/ | ie (A) − | minus (A) or |
| Treatment | plant | (B)/(A) × 100 | (B)/(C × 100 |
| Control (C) | 2.7 | — | — |
| AC | 2.7 | — | — |
| Anthranilic acid (A) | 2.2 | — | +18.5 |
| Anthranilic acid + AC (B) | 1.5 | +31.8 | +44.4 |
| Anthranilic acid methyl ester | 2.2 | — | +18.5 |
| (A) | | | |
| Anthranilic | 1.7 | +22.7 | +37.0 |
| acid methyl ester + AC (B) | | | |
| Acetamide (A) | 2.3 | — | +14.8 |
| Acetamide + AC (B) | 1.9 | +17.4 | +29.6 |
| Anthranilic Acid monosodium | 2.3 | — | +14.8 |
| salt (A) | | | |
| Anthranilic Acid monosodium | 1.7 | +26.1 | +37.0 |
| salt + AC (B) | | | |
| Indole-3-acetamide (A) | 2.3 | — | +14.8 |
| Indole-3-acetamide + AC (B) | 1.5 | +34.8 | +44.4 |
| 3-Hydroxyanthranilic acid (A) | 2.5 | — | +7.4 |
| 3-Hydroxyanthranilic acid + | 2.2 | +12.0 | +18.5 |
| AC (B) | | | |
| Tryptamine (A) | 2.7 | — | +0.0 |
| Tryptamine + AC (B) | 2.3 | +14.8 | +14.8 |
| L-Tryptophan (A) | 2.4 | — | +11.1 |
| L-Tryptophan + AC (B) | 2.0 | +16.7 | +25.9 |

Application of an auxin or auxin precursor resulted in a reduced mean number of buds per plant, as expected from an effective thinning agent. Application of AC in addition to an auxin or auxin precursor resulted in further reduction in mean number of buds per plant.

B. To demonstrate the benefits to flower bud thinning of applying agrochemically acceptable additives in combination with either anthranilic acid or anthranilic acid plus acetaminophen

Cultural Details: plants were sown in a commercial seed compost and then “pricked out” into a professional grade all-purpose compost in 9 cm pots set out in a randomized block design. Normal daylight plus supplementary lighting to provide a minimum of 10 hours per day. Heating was given to a minimum of 60 degrees Fahrenheit during the day and 40 degrees at night. Certain agrochemically acceptable additives, as examples (rates per ha provided in the Table below), were added to either anthranilic acid (1 gram per litre solution, applied in 250 litres water per hectare, =1 g active total per hectare) or anthranilic acid plus acetaminophen (1 gram per litre and 3 gram per litre solution respectively, applied in 250 litres water per hectare, =1 g and 3 g actives total respectively per hectare).

Sowing Date: 26 Oct. 2009, in plugs. Application Date: 4 Feb. 2010 (first bud). Measurement Date: 12 Mar. 2010 (flowering). Measurement: Number of buds per plant.

| | Mean | | % Improvement |
|---|---|---|---|
| | Number | % | over appropriate |
| | of | Improvement | control (ie AN or |
| Treatment | Buds/plant | over untreated | AN + AC) |
| Untreated | 2.8 | — | — |
| Acetaminophen (AC) | 2.8 | — | — |
| Anthranilic acid (AN) | 2.3 | +17.8 | — |
| AN + AC | 1.9 | +32.1 | +17.4 |
| AN + Agrochemically | 2.1 | +25.0 | +8.7 |
| acceptable additive | | | |
| (AAA): Glucose 150 g | | | |
| (category (a) example) | | | |
| AN + AC + | 1.7 | +39.3 | +10.5 |
| Agrochemically | | | |
| acceptable additive | | | |
| (AAA): Glucose 150 g | | | |
| (category (a) example) | | | |
| AN + Agrochemically | 2.1 | +25.0 | +8.7 |
| acceptable additive | | | |
| (AAA): Succinic acid | | | |
| 50 g (category (b) | | | |
| example) | | | |
| AN + AC + | 1.7 | +39.3 | +10.5 |
| Agrochemically | | | |
| acceptable additive | | | |
| (AAA): Succinic acid | | | |
| 50 g (category (b) | | | |
| example) | | | |
| AN + Agrochemically | 2.0 | +28.6 | +13.0 |
| acceptable additive | | | |
| (AAA): Ascorbic acid | | | |
| 50 g (category (c) | | | |
| example) | | | |
| AN + AC + | 1.7 | +39.3 | +10.5 |
| Agrochemically | | | |
| acceptable additive | | | |
| (AAA): Ascorbic acid | | | |
| 50 g (category (c) | | | |
| example) | | | |
| AN + Agrochemically | 2.0 | +28.6 | +13.0 |
| acceptable additive | | | |
| (AAA): Corn oil 500 g | | | |
| (category (e) example) | | | |
| AN + AC + | 1.65 | +41.1 | +13.2 |
| Agrochemically | | | |
| acceptable additive | | | |
| (AAA): Corn oil 500 g | | | |
| (category (e) example) | | | |
| AN + Agrochemically | 2.1 | +25.0 | +8.7 |
| acceptable additive | | | |
| (AAA): L-arginine 30 g | | | |
| (category (f) example) | | | |
| AN + AC + | 1.7 | +39.3 | +10.5 |
| Agrochemically | | | |
| acceptable additive | | | |
| (AAA): L-arginine 30 g | | | |
| (category (f) example) | | | |

Application of an agrochemically acceptable additive in combination with AN shows a further improvement over AN alone. Similarly, application of an agrochemically acceptable additive in combination with AN+AC shows a further improvement over AN+AC alone.

All publications mentioned in the above specification are herein incorporated by reference. Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the field are intended to be within the scope of the following claims.

1-33. (canceled)

34. A composition comprising a compound selected from an auxin, an auxin precursor, an auxin metabolite or a derivative of said auxin, auxin precursor or auxin metabolite or a mixture thereof and acetaminophen or a derivative thereof for use as a chemical thinning agent.

35. A composition comprising a compound selected from an auxin, an auxin precursor, an auxin metabolite or a derivative of said auxin, auxin precursor or auxin metabolite or a mixture thereof, acetaminophen or a derivative thereof and an agrochemically acceptable additive for use as a chemical thinning agent.

36. The composition of claim 35 wherein the agrochemically acceptable additive comprises at least one compound selected from:
a) glucose, hydrolysed starch, sucrose, fructose, glycerol, glyceraldehydes, erythrose, ribulose, xylulose or arabinose, monosaccharides including aldoses such as D-Ribose, D-Xylose, L-Arabinose, D-Glucose, D-Mannose and D-Galactose; ketoses such as D-Ribulose and D-Fructose; deoxyaldoses such as 2-Deoxy-D-ribose, L-Fuccose; acetylated amino sugars such as N-Acteyl-D-glucosamine and N-Acetyl-D-galactosamine; acidic monosaccharides such as D-Glucuronic acid, L-Iduronic acid and N-Acetylneuraminic acid, Sugar alcohols such as D-Sorbitol and D-Mannitol, disaccharides including maltose, lactose and sucrose, or an ester or glycoside or metabolic equivalent of such a carbohydrate;
b) an organic acid of the Krebs tricarboxylic acid cycle or a metabolic precursor thereof;
c) a vitamin or coenzyme, or a precursor thereof;
d) a purine or pyrimidine nucleoside, nucleotide or metabolic precursor thereof;
e) a naturally occurring fat or oil; or
f) an amino acid.

37. The composition of claim 36 wherein the agrochemically acceptable additive comprises at least one compound selected from c) a vitamin or coenzyme, or a precursor thereof; d) a purine or pyrimidine nucleoside, nucleotide or metabolic precursor thereof; or f) an amino acid.

38. The composition of claim 34 wherein the composition further comprises an adjuvant.

39. The composition according to claim 34 wherein the auxin is an indolic auxin or a phenolic auxin.

40. The composition according to claim 34 wherein the auxin derivative is an acid, a conjugate, a salt, an ester, or an amide of the auxin, or an alkylated or halogenated auxin.

41. The composition according to claim 40 wherein the auxin is conjugated to a sugar, an alcohol, an amino acid, a peptide or a protein.

42. The composition according to claim 34 wherein the precursor is chorismate, anthranilic acid, phosphoribosyl anthraniliate, 1-(O-carboxyphenulamino)-1-deoxyribulose-5-phosphate, indole-3-glycerol-phosphate, indole, indole-3-acetic acid, tryptophan, tryptamine, N-hydroxy tryptamine, indole-3-acetaldoxime, 1-aci-nitro-2-indolylethane, indolic glucosinate, indole-3-acetonitrile (IAN), indole-3-acetaldehyde, indole-3-lactic acid, indole-3-pyruvic acid, or indole-3-ethanol; preferably anthranilic acid.

43. The composition according to claim 34 wherein the auxin is a natural or synthetic auxin.

44. The composition according to claim 34 wherein the auxin is a natural auxin selected from indole-3-acetic acid (IAA), 4-chloro-indole-3-acetic acid (4-Cl-IAA), phenylacetic acid (PAA), indole-3-butyric acid (IBA), indole-3-acetyl-1-O-β-D-glucose (IAAglc); or a synthetic auxin selected from 1-naphthaleneacetic acid (NAA), 2,4-dichlorophenoxyacetic acid (2,4-D), 2-methoxy-3,6-dichlorobenzoic acid (dicamba), 4-amino-3,5,6-trichloropicolinic acid (tordon), 2,4,5-trichlorophenoxyacetic acid (2,4,5-T), 2,3,6-trichlorobenzoic acid, 4-chloro-2 methyl phenoxy acetic acid (MCPA), N,N-dimethylethylthiocarbamate or napthaleneacetamide NAD).

45. The composition according to claim 44 wherein the conjugate of the natural auxin is IAA-Inositol, IAA-Inositol-arabinose, IAP1, an IAA-peptide, an IAA glycoprotein, an IAA-glucan, IAA-aspartate, IAA-glucose, IAA-1-O-glucose, IAA-myo-Inositol, IAA-4-O-glucose, IAA-6-O-glucose, IAA-Inositol-galactose, an IAA amide conjugate, or an IAA-amino acid conjugate.

46. Anthranilic acid or a derivative thereof for use as a chemical thinning agent.

47. A composition comprising two or more of the compounds of claim 46 for use as a chemical thinning agent.

48. A composition comprising a compound selected from an auxin, an auxin precursor, an auxin metabolite or a derivative of said auxin, auxin precursor or auxin metabolite or a mixture thereof and an agrochemically acceptable additive for use as a chemical thinning agent.

49. The composition according to claim 48 wherein the agrochemically acceptable additive comprises at least one compound selected from:
a) glucose, hydrolysed starch, sucrose, fructose, glycerol, glyceraldehydes, erythrose, ribulose, xylulose or arabinose, monosaccharides including aldoses such as D-Ribose, D-Xylose, L-Arabinose, D-Glucose, D-Mannose and D-Galactose; ketoses such as D-Ribulose and D-Fructose; deoxyaldoses such as 2-Deoxy-D-ribose, L-Fuccose; acetylated amino sugars such as N-Acteyl-D-glucosamine and N-Acetyl-D-galactosamine; acidic monosaccharides such as D-Glucuronic acid, L-Iduronic acid and N-Acetylneuraminic acid, Sugar alcohols such as D-Sorbitol and D-Mannitol, disaccharides including maltose, lactose and sucrose, or an ester or glycoside or metabolic equivalent of such a carbohydrate;
b) an organic acid of the Krebs tricarboxylic acid cycle or a metabolic precursor thereof;
c) a vitamin or coenzyme, or a precursor thereof;
d) a purine or pyrimidine nucleoside, nucleotide or metabolic precursor thereof;
e) a naturally occurring fat or oil; or
f) an amino acid.

50. A composition for use as a chemical thinning agent comprising a composition of claim 34 in combination with a further chemical thinning agent; wherein the further chemical thinning agent is preferably selected from benzyladenine, 1-naphthylacetic acid, carbaryl, (2-chlorophenoxy)propionic acid, ethephon, naphthaleneacetamide, thidiazuron, ammonium thiosulphate, DNOC, endothallic acid, gibberellic acid, lime sulphur, sulfcarbamide and pelargonic acid.

51. The composition of claim 34 for use as a fruitlet thinner or a flower thinner.

52. A method of chemical thinning comprising applying the composition of claim 34 to a plant or its environs, wherein the plant preferably is a tree, shrub, vine vegetable or other crop, or an ornamental.

53. A method of thinning fruitlets or flowers comprising applying the composition of claim 34 to a plant or its environs, wherein the plant preferably is a tree, shrub, vine, vegetable or other crop, or an ornamental.
